(19) United States
(12) Patent Application Publication
(10) Pub. No.: US 2014/0079595 A1
(43) Pub. Date: Mar. 20, 2014

(54) EMBEDDED MULTIFUNCTIONAL AIR PURIFIER USED IN KITCHEN

(71) Applicants: CHING-CHENG KAO, NEW TAIPEI CITY (TW); CHING-HUNG CHIEN, NEW TAIPEI CITY (TW)

(72) Inventors: CHING-CHENG KAO, NEW TAIPEI CITY (TW); CHING-HUNG CHIEN, NEW TAIPEI CITY (TW)

(73) Assignee: KOMPASS TECHNOLOGY CO., LTD., NEW TAIPEI CITY (TW)

(21) Appl. No.: 13/616,063

(22) Filed: Sep. 14, 2012

(51) Int. Cl. A61L 9/00

(52) U.S. Cl. 422/121; 422/120

(57) ABSTRACT

An embedded multifunctional air purifier used in a kitchen is installed in a predetermined space of a cupboard and includes a casing, a front filter, an electrostatic dust collector, an air extraction module, a photocatalyst module and a vitamin C filter. An air inlet and an air outlet are formed on a front side of the casing; an air channel is formed in the casing; the front filter is installed at the air inlet; the electrostatic dust collector id installed at a front section of the air channel; the air extraction module and the photocatalyst module are installed at a rear section of the air channel; and the vitamin C filter is installed at the air outlet, so as to eliminate fume odor and germs in the kitchen and provides the function of producing vitamin C.

### BACKGROUND OF THE INVENTION

1. Field of the Invention

The present invention relates to the technical field of air purifiers, and more particularly to an embedded multifunctional air purifier used in a kitchen.

2. Description of the Related Art

As health becomes increasingly more important nowadays, many people gradually abandon the rule of having the three regular meals everyday and attempt to cook health food in a kitchen instead. Ironically, these people improve their nutrition by the health food, but also accidentally step in another intangible risk or hidden worry of having lung cancer. For example, carbon monoxide, poly-cyclic aromatic smoke, hetero-cyclic amine, nitro-polycyclic aromatic smoke and cigarette smoke in the kitchen fume are high-risk carcinogens.

To reduce or eliminate fumes, a range hood is generally installed in a kitchen. However, the range hood always has the issue of insufficient air extraction power and only can extract instantaneously produced fumes, and thus the range hood is simply used for a general open cooking utility. For steamers or ovens, such range hood is not applicable. In the kitchen space, dirty air particles are produced all the time, instead of during the cooking process only. In addition, the pollution caused by kitchen wastes or garbage must be taken into consideration as well. Since the kitchen wastes have a high water content (generally over 85%) and most of them are easily rotten smell substances, therefore kitchen wastes create a ground of breeding bacteria and causing diseases. Therefore, housewives staying in such environment fir a long time belong to high-risk group of cancer due to the fumes. In addition, the bacteria in the kitchen wastes may cause diseases easily, and the skin of housewives may become aged easily under the condition of exposing to the fume for a long time.

In view of the aforementioned problems, the inventor of the present invention provides an embedded multifunctional air purifier used in a kitchen, not only providing an easy installation of the air purifier in the kitchen environment, but also eliminating fumes effectively. Meanwhile, a vitamin C filter is provided for inhibiting bacteria, improving human immunity and preventing skin ageing, so as to provide a healthy and safe environment for users or housewives who stay in a kitchen for a long time.

### SUMMARY OF THE INVENTION

In view of the aforementioned problems, it is a primary objective of the present invention to provide a multifunctional air purifier capable or eliminating fume and odor while providing anions and releasing vitamin C particles into air to achieve the effects of improving human immunity, preventing skin ageing, and allowing users or housewives staying in a kitchen for long time to have a healthy and safe environment.

To achieve the foregoing objective, the present invention provides an embedded multifunctional air purifier used in a kitchen, wherein the air purifier is installed in a predetermined space of a cupboard without requiring an additional installation of an air guide pipeline, so that the installation of the air purifier becomes very convenient and the installed air purifier looks aesthetic. In addition, a liftable cover plate and a slide rail are added to the design to facilitate users to remove, maintain, repair and clean the air purifier, so as to enhance the convenience of use significantly.

To achieve the aforementioned objective, the present invention provides an embedded multifunctional air purifier used in a kitchen, comprising: a casing, having an air inlet and an air outlet formed on a front side of the casing, and an air channel formed in the casing, and both end surfaces of the air channel being coupled to the air inlet and the air outlet respectively, and a liftable cover plate being installed at the top of the casing; a front filter, installed at the air inlet; an electrostatic dust collector, installed at a front section of the air channel; an air extraction module, installed at a rear section of the air channel, for sucking an airflow from the air inlet to the air outlet through the air channel; a photocatalyst module, installed at the rear section of the air channel; and a vitamin C filter, installed at the air outlet.

In the present invention, the photocatalyst module includes an UV sterilization lamp and a photocatalyst filter. To enhance the isolation effect of the front filter, a three-layer cross aluminum net structure is adopted. To eliminate odor, an activated carbon filter is installed at a rear section of the air channel.

In addition, the multifunctional air purifier of the present invention is embedded into a kitchen wall of a container. To facilitate controlling the air purifier, the air purifier further comprises a wireless receiving device, for receiving a wireless signal to electrically turn on or off an operation of the air purifier. In addition, an anion machine is installed at the easing for releasing anions.

The air extraction module of the present invention includes a fixing board and a pair of fans, and the fans are arranged parallely on the fixing board to enhance the air flow significantly, while slightly increasing the noise value.

Therefore, the present invention can achieve the effects of isolating and collecting the dust, powder, fume and dust mites in air and decomposing the allergens and mildews in air to reduce the occurrence of allergic diseases. The invention also can inhibit and eliminate odor effectively. After the filtering process takes place, clean air released to the kitchen also contains vitamin C in the air particles, and vitamin C can moisten the users' skin and preventing skin ageing.

### BRIEF DESCRIPTION OF THE DRAWINGS

FIG. 1 is a schematic view of an installation of a preferred embodiment of the invention;

FIG. 2 is a perspective view of a preferred embodiment of the invention;

FIG. 3 is an exploded view of a preferred embodiment of the invention; and

FIG. 4 is a schematic view of the circulation of air during use in accordance with a preferred embodiment of the present invention.

### DETAILED DESCRIPTION OF THE PREFERRED EMBODIMENTS

The technical contents of the present invention will become apparent with the detailed description of preferred embodiments and the illustration of related drawings as follows.

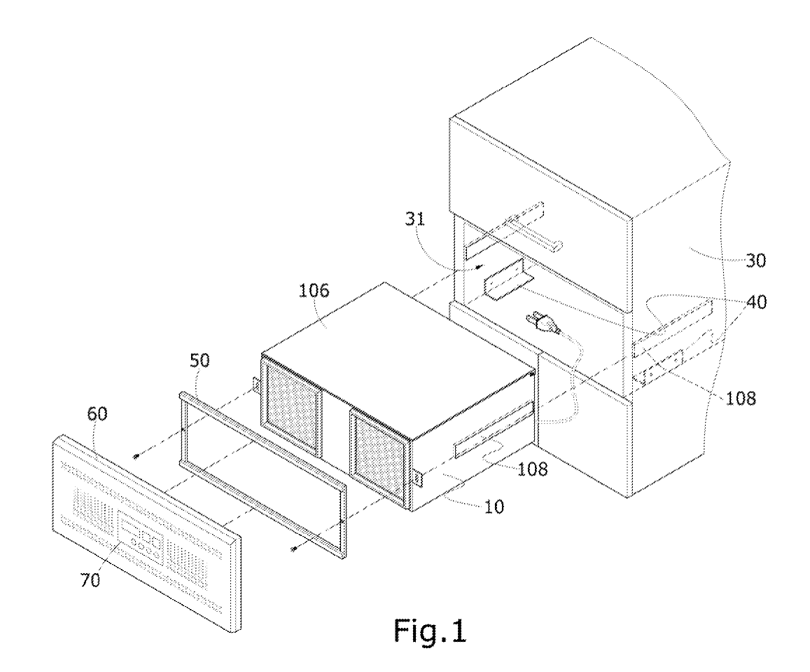

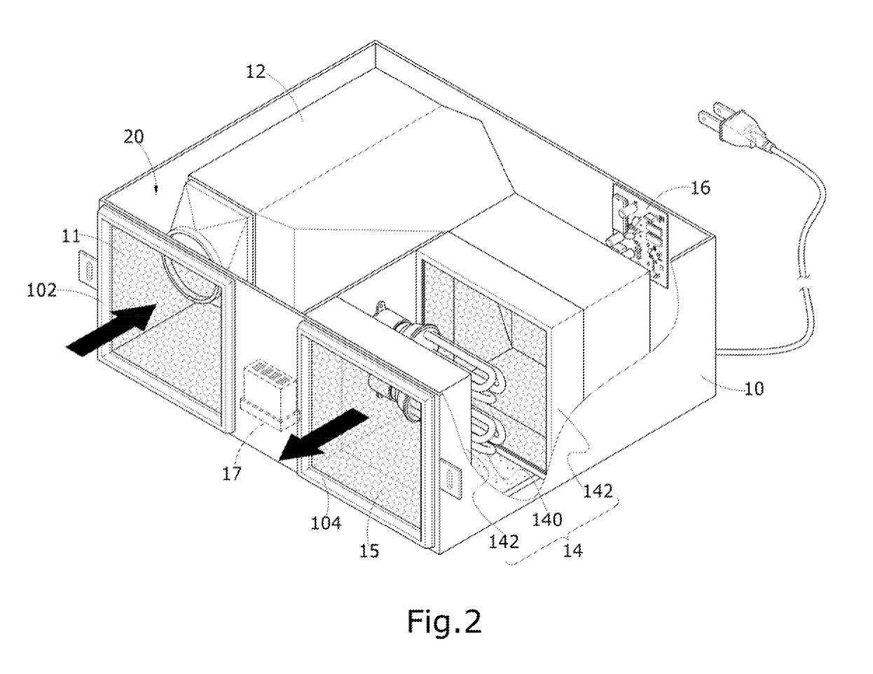

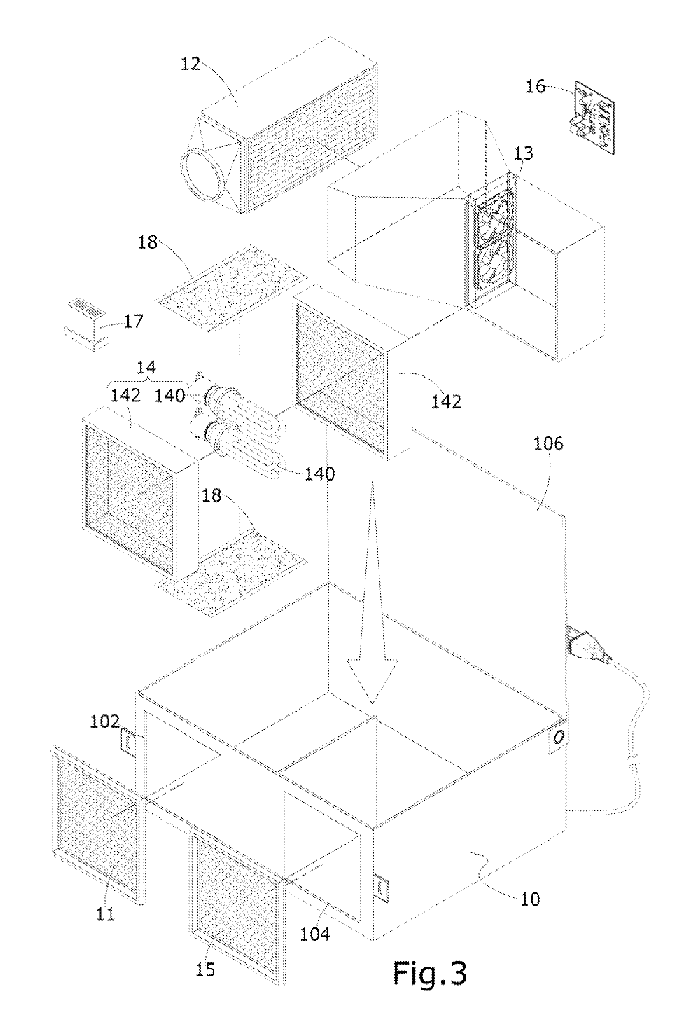

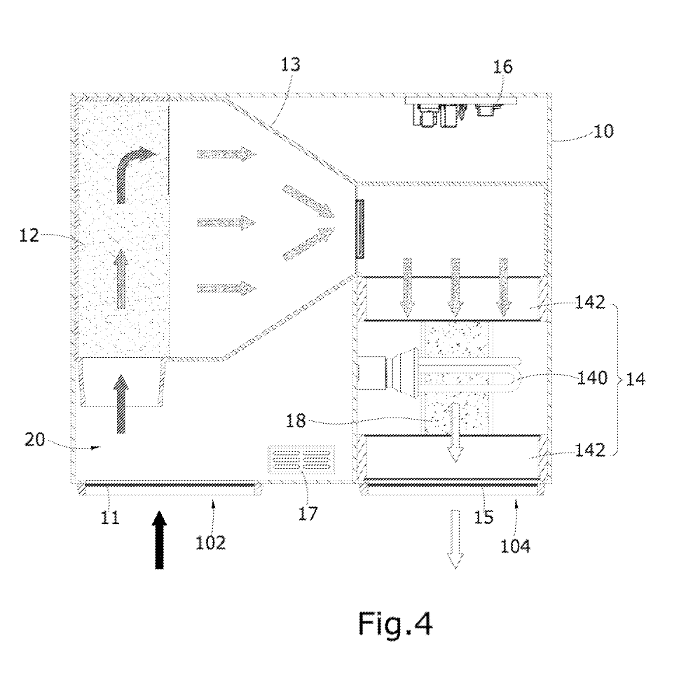

With reference to FIGS. 1 to 4 for a schematic view of the installation, a perspective view, an exploded view, and a schematic view of the circulation of air during use in accordance with a preferred embodiment of the present invention respectively, the present invention discloses an embedded multifunctional air purifier used in a kitchen and installed in a predetermined space 31 of a cupboard 30. The air purifier comprises: a casing 10, a front filter 11, an electrostatic dust collector 12, an air extraction module 13, a photocatalyst module 14 and a vitamin C filter 15. The casing 10 is substantially in the shape of a square box. After the casing 10 is embedded into a kitchen cabinet or a wall, the casing 10 in contact with a surface of the environment has an air inlet 102 and an air outlet 104, and an air channel 20 is formed in the casing 10, and a slide rail 106 is coupled separately to both surfaces of a front side of the casing 10, and a section of the pair of slide rails 106 is fixed onto the casing 10 for connecting to the predetermined space 31, so that the casing 10 can be moved linearly with respect to the predetermined space 31. In addition, a liftable cover plate 108 is installed at the top of the casing 10, so that when a user wants to maintain, repair or clean the air purifier, the user pulls the casing 10 out along the extension direction of the slide rail 106 and then lifts the liftable cover plate 108 to perform various different operations quickly. Both ends of the air channel 20 are coupled to the air inlet 102 and the air outlet 104 respectively. In other words, air particles in a kitchen environment are entered into the casing 10 through the air inlet 102, and then passed into the air channel 20, and finally discharged from the air outlet 104 and returned into the kitchen environment. Since the air flowing in the air channel 20 will pass through different filtering or purifying devices such as the front filter 11, the electrostatic dust collector 12, the air extraction module 13, the photocatalyst module 14 and the vitamin C filter 15 or even an anion machine 17 for improving the purification of the air particles and the quality of air, or an activated carbon filter 18 installed at the rear section of the air channel 20 for removing odor in the air, clean air with a high quality can be obtained. The aforementioned operation is repeated to achieve a clean air environment. In addition, the multifunctional air purifier of the present invention is embedded in a kitchen wall or a container. To facilitate controlling the air purifier, the air purifier further comprises a wireless receiving device 16 for receiving a wireless signal to electrically turn on or off an operation of the air purifier.

It is noteworthy that the air extraction module 13 includes a fixing board 132 and a pair of fans 134 to enhance the air input, wherein the fans 134 are installed parallely on the fixing board 132 and behind the air channel 20 to enhance the air flow significantly while slightly increasing noise value by approximately 2 dB to 3 dB.

Further, the multifunctional air purifier of the present invention can be installed in a cupboard 30 or a wall, and a pair of fixing racks 40 is provided for supporting and fixing the casing 10. A magnetic acrylic board 50 can be attached onto surfaces of the air inlet 102 and the air outlet 104 of the multifunctional air purifier casing 10 which is embedded into the cupboard or the wall. For example, the magnetic acrylic board 50 can be fixed by locking or any other fixing method. Finally, a stainless steel panel 60 is magnetically attached onto the magnetic acrylic board 50, so as to complete the installation procedure of the multifunctional air purifier.

During the air purification process, air particles in the kitchen are passed through the front filter 11 installed at the air inlet 102 to isolate the dust with a larger size in air by a direct physical penetrating method. To improve the isolation effect, the front filter 11 is of a three-layer cross aluminum net structure capable of preventing the large-sized air molecules from passing through. And then, the air particles are passed through the electrostatic dust collector 12 installed at a front section of the air channel 20 to pass the particles from the front filter 11 to an electric charge zone and drive the particles to carry positive and negative charges. And then, an electric field is provided for attaching and collecting the larger particles to achieve the electrostatic dust collection effect. Particularly, such technology can collect fine dust, mites and powder with a size of 0.2 micron.

To improve the air circulation efficiency, an air extraction module 13 is installed at a rear section of the air channel 20 inside the casing 10 of the multifunctional air purifier, wherein the air extraction effect of the air extraction module 13 can accelerate the suction of the air particles remained in the kitchen into the air purifier, in order to suck the micro-particulates passing through the front filter 11 and the electrostatic dust collector 12 quickly through the air extraction module 13 and guide the particulates to the next cleaning device. In the aforementioned cleaning process, the photocatalyst module 14 is further installed at a rear section of the air channel 20, wherein the photocatalyst module 14 is comprised of an UV sterilization lamp 140 and a photocatalyst filter 142. Since the photocatalyst can be excited by the UV light to produce very active peroxide molecules, superoxide molecules, and free-radical oxide molecules to decompose organic gases and odors into carbon dioxide and water. In addition, when micro-organisms are in contact with the surface of the photocatalyst, the superoxide molecules will damage the membrane of the micro-organism to kill the micro-organisms. In addition, the photocatalyst filter 142 can degenerate the attached air molecules continuously, re-circulate the air and re-decompose air particles, so as to achieve the permanent air cleaning effect. Unlike the general activated carbon surface which can adsorb odor up to a saturation level only and requires a replacement from time to time, the present invention can provide a permanent odor removal effect.

When the processed air molecules are outputted from the air outlet 104, the air molecules pass through the vitamin C filter 15 installed at the air outlet 104, and the vitamin C filter 15 can release vitamin C into the air to achieve the effects of moistening and beautifying the skin of users and inhibiting skin ageing. In addition, the anion machine 17 is provided for releasing anions to further refresh the processed air.

In summation of the description above, the embedded multifunctional air purifier used in a kitchen in accordance with the present invention can achieve the effects of isolating and collecting the dust, powder, fume and dust mites in air and decomposing the allergens and mildews in the air to reduce the occurrence of allergic diseases. The present invention also can inhibit and eliminate odors effectively, Clean air is released into the kitchen again after the filtering process takes place. Meanwhile, a vitamin C filter is provided for inhibiting bacteria, improving human immunity and preventing skin ageing, so as to provide a healthy and safe environment for users or housewives who stay in a kitchen for a long time.

While the invention has been described by means of specific embodiments, numerous modifications and variations could be made thereto by those skilled in the art without departing from the scope and spirit of the invention set forth in the claims.

In summation of description above, the present invention improves over the prior art and complies with patent application requirements, and thus is duly filed for patent application.

What is claimed is:

1. An embedded multifunctional air purifier used in a kitchen, and installed in a predetermined space of a cupboard without requiring an additional installation of an air guide pipeline, comprising:
a casing, having an air inlet and an a outlet formed on a front side of the casing, and an air channel formed in the casing, and both end surfaces of the air channel being coupled to the air inlet and the air outlet respectively, and a liftable cover plate being installed at the top of the casing;
a front filter, installed at the air inlet;
an electrostatic dust collector, installed at a front section of the air channel;
an air extraction module, installed at a rear section of the air channel, for sucking an airflow from the air inlet to the air outlet through the air channel;
a photocatalyst module, installed at the rear section of the air channel; and
a vitamin C filter, installed at the air outlet.

2. The air purifier of claim 1, wherein the photocatalyst module includes an UV sterilization lamp and a photocatalyst filter.

3. The air purifier of claim 2, wherein the front filter is a three-layer cross aluminum net structure.

4. The air purifier of claim 1, further comprising: a wireless receiving device, for receiving a wireless signal to electrically turn on or off an operation of the air purifier.

5. The air purifier of claim 1, further comprising an anion machine installed in the casing for releasing anions.

6. The air purifier of claim 1, further comprising a magnetic acrylic board and a stainless steel panel, and the magnetic acrylic board being coupled to a front side of the casing, and the stainless steel panel is magnetically coupled to the acrylic board.

7. The air purifier of claim 6, further comprising a pair of fixing racks for supporting and fixing the casing.

8. The air purifier of claim 1, further comprising a pair of slide rails disposed on both opposite sides of the casing respectively to couple the predetermined space.

9. The air purifier of claim 1, wherein the air extraction module includes a fixing board and a pair of fans, and the pair of fans are arranged parallelly on the fixing board.

10. The air purifier or claim 1, further comprising an activated carbon filter installed at the air channel rear section and provided for removing odor.
